(19) United States
(12) Patent Application Publication
(10) Pub. No.: US 2019/0347786 A1
(43) Pub. Date: Nov. 14, 2019

(54) METHOD, APPARATUS, AND ELECTRONIC DEVICE HAVING LIVING BODY DETECTION CAPABILITY

(71) Applicant: PixArt Imaging Inc., Hsin-Chu City (TW)

(72) Inventors: Chih-Hsin Lin, Hsin-Chu City (TW); Ren-Hau Gu, Hsin-Chu City (TW); Chien-Lung Liao, Hsin-Chu City (TW); Yi-Fang Lee, Hsin-Chu City (TW)

(21) Appl. No.: 15/973,553

(22) Filed: May 8, 2018

(51) Int. Cl. G06T 7/00; A61B 5/00; A61B 5/1172

(52) U.S. Cl. CPC G06T 7/0012; G06K 9/00114; A61B 5/1172; A61B 5/0077

(57) ABSTRACT

A method of living body detection includes: capturing a plurality of images of an object under test by using an image sensor; calculating variations of brightness values of pixel units of the plurality of images; and, determining whether the object under test is a living body according to the variations.

### BACKGROUND OF THE INVENTION

### 1. Field of the Invention

The invention relates to an image sensing mechanism, and more particular to a method, an apparatus, and an electronic device having living body detection capability based on result(s) of an image sensor.

### 2. Description of the Prior Art

Generally speaking, a conventional living body detection scheme may be arranged to perform one complete operation of the living body detection by using a sensor to measure a Photoplethysmography (PPG) signal for collecting reliable heart rate data. Since a human′ heart rate may range from 60 bpm to 100 bpm (beats per minute), for collecting the reliable heart rate data for a human, it is needed to at least measure a complete Cardiac cycle for one time and thus the conventional scheme may need to wait for at least 0.6 s (600 ms) or one second (1000 ms) for one complete operation of the living body detection. The performance of conventional living body detection scheme now becomes not accepted by users especially for fake fingerprint detection.

### SUMMARY OF THE INVENTION

Therefore one of the objectives of the invention is to provide a method, apparatus, and electronic device having novel living body detection capability, to solve the above-mentioned problems.

According to embodiments of the invention, a method of living body detection is disclosed. The method comprises: capturing a plurality of images of an object under test by using an image sensor; calculating variations of brightness values of pixel units of the plurality of images; and, determining whether the object under test is a living body according to the variations.

According to the embodiments, an apparatus having living body detection capability is disclosed. The apparatus comprises an image sensor and a processor. The image sensor is configured for capturing a plurality of images of an object under test. The processor is coupled to the image sensor and is configured for calculating variations of brightness values of pixel units of the plurality of images, and for determining whether the object under test is a living body according to the variations.

According to the embodiments, an electronic device is disclosed. The electronic device comprises a physiological feature detection circuit, an image sensor, and a processor. The image sensor is configured for capturing a plurality of images of an object under test. The processor is coupled to the image sensor and is configured for calculating variations of brightness values of pixel units of the plurality of images and for determining whether the object under test is a living body according to the variations. The processor is arranged to disable the physiological feature detection circuit when the object under test is determined as a non-living body.

According to the embodiments, another electronic device is disclosed. The electronic device comprises a physiological feature detection circuit, an image sensor, and a processor. The image sensor is configured for capturing a plurality of images of an object under test. The processor is coupled to the image sensor and is configured for calculating variations of brightness values of pixel units of the plurality of images and for determining whether the object under test is a living body according to the variations. The processor is arranged to stop outputting a physiological feature when the object under test is determined as a non-living body.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a flowchart of a method of living body detection according to embodiments of the invention.

FIG. 2 is a block diagram of an apparatus having living body detection capability according to the embodiments of FIG. 1.

FIG. 3 is an example respectively shows a difference comparison between variations of brightness of image sensed from a living body and variations of brightness of image sensed from a non-living body.

FIG. 4 is a block diagram of an apparatus having living body detection capability according to another embodiment of FIG. 1.

### DETAILED DESCRIPTION

The invention aims at providing a sensing scheme/mechanism capable of effectively and accurately determining whether an object under test is a living body or a non-living body based on a result outputted by an image sensor.

The sensing scheme/mechanism may be implemented for a portable apparatus having a processor and the image sensor. In one embodiment the image sensor may emit light rays to the object under test and detect the light reflections from or passing through the object under test. The image sensor could be a general/specific image sensor.

For example, the portable apparatus may be comprised by an electronic device such as a wearable electronic device with the physiological feature detection capability. For instance, such electronic device may be a smart bracelet/watch device (but not limited). When a user wears such smart bracelet/watch device, the object under test may indicate the user's wrist, i.e. a living body.

However, if the smart bracelet/watch device is not wore by the user and placed on a working desk, the object under test may become the top surface of the desk, i.e. a non-living body. The sensing scheme/mechanism can effectively detect whether the object under test is a living body or not. It is to be noted that the type of the object under test is not meant to be a limitation. In other applications, the object under test may mean a human's finger or other limbs or body parts. In addition, the sensing scheme/mechanism may be arranged to detect whether the object under test is an alive animal (a living body) or not.

It should be noted that in the embodiments a living body may indicate a body having blood flow while a non-living body may indicate a body not having blood flow. For instance, a stone material, an metal material, a wood material, or a part of a dead animal/body may be a non-living body since the materials do not have blood flow and the dead part has not have blood flow.

For example, the sensing scheme/mechanism can be arranged to rapidly and effectively determine a fake fingerprint as a non-living body. For optical fingerprint unlock applications, the sensing scheme/mechanism can raise the information security protection significantly.

In addition, since the sensing scheme/mechanism of the invention can be performed merely based on result(s) of a generic image sensor, the circuit and computation costs can be reduced.

The sensing scheme/mechanism can be activated before activating a physiological feature detection circuit, so that the result of sensing scheme/mechanism can be provided to determine whether to activate or enable the physiological feature detection circuit. For example, if the result of sensing scheme/mechanism shows a non-living body, then the physiological feature detection circuit will not be enabled since it is unnecessary to detect the physiological feature of a non-living body.

In one embodiment, the physiological feature detection circuit is enabled only when the result of sensing scheme/mechanism shows a living body. By doing so, the physiological feature detection circuit can be appropriately enabled/disabled to save more power of an electronic device and more particularly to a portable electronic device powered by a battery.

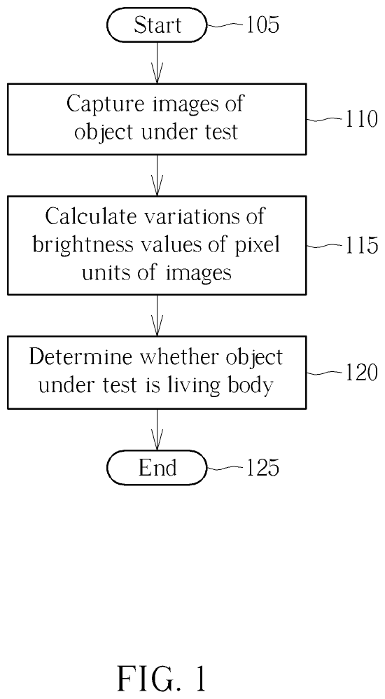

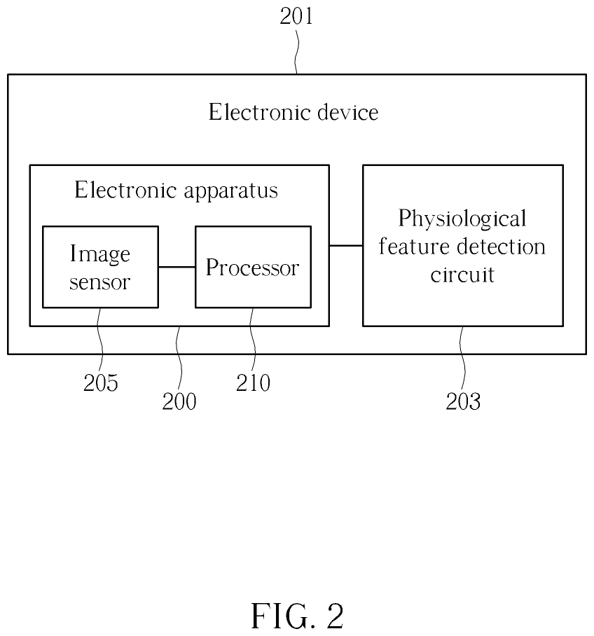

Refer to FIG. 1, which is a flowchart of a method of living body detection according to embodiments of the invention. FIG. 2 is a block diagram of an apparatus having living body detection capability according to the embodiments of FIG. 1.

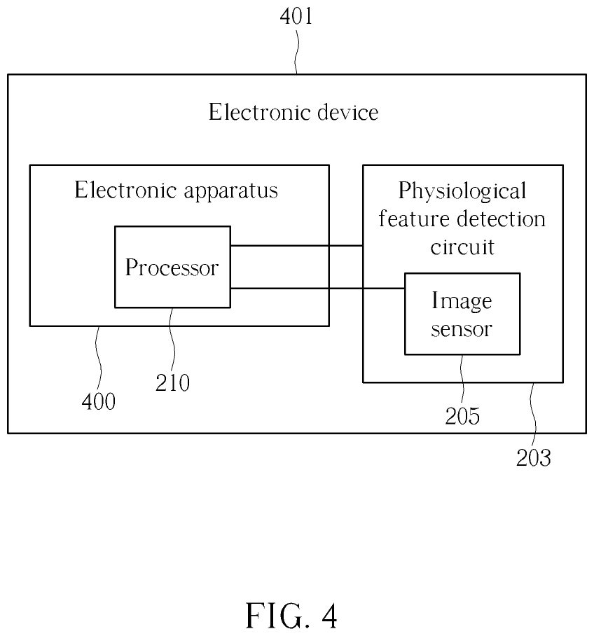

The apparatus 200 comprises an image sensor 205 and a processor 210. In another embodiment, the image sensor 205 may be a portion of a physiological feature detection circuit 203; this example is shown on FIG. 4.

The apparatus 200 of FIG. 2 for example is included within an electronic device 201 which may further comprise a physiological feature detection circuit 203 such as a hear rate detection circuit or a blood pressure detection circuit; however, this is not a limitation. The electronic device 201 may be a portable electronic device (but not limited) such as a wearable electronic device with the physiological feature detection capability of circuit 203. For instance, such electronic device may be a smart bracelet/watch device (but not limited).

Provided that substantially the same result is achieved, the steps of the flowchart shown in FIG. 1 need not be in the exact order shown and need not be contiguous, that is, other steps can be intermediate. Steps are detailed in the following:

Step 105: Start;

Step 110: Capture and obtain a plurality of images of the object under test;

Step 115: Calculate variations of brightness values of pixel units of the images;

Step 120: Determine whether the object under test is a living body according to the calculated variations; and

Step 125: End.

In Step 110, for example, the image sensor 205 is arranged to capture and obtain a plurality of images of the object under test. For example, the object under test may be a living body such as a human's finger (but not limited) or a non-living body such as a stone or a desktop (but not limited). The image sensor 205 for example may be implemented by using a generic image sensor to save circuit costs or may be implemented a specific/advanced image sensor for specific purposes. This is not meant to be a limitation.

In addition, the frame rate of image sensor 205 may be equal to 120 frames per second (but not limited), and the processor 210 may receive a predetermined number of successive frames from the image sensor 205 to determine a living body. For example, the number of frames/images provided by the image sensor 205 to the processor 210 for living body detection may be equal to ten. However, this is not intended to be a limitation. The needed number of frames/images for the living body detection can be modified or configured by the user.

Based on the frames/images provided from the image sensor 205, the processor 210 in Step 115 is able to perform the living body detection. In practice, the processor 210 is arranged to calculate variations of brightness values of pixel units of the plurality of images and then to determine whether the object under test is a living body according to the variations. The processor 210 can compare the variations with a threshold to determine whether the object under test is a living body.

In the following descriptions of the embodiments, a pixel unit is configured to comprise a single one pixel. That is, for each pixel, the processor 210 calculates variations of the brightness values on the plurality of images and then to determine whether the object under test is a living body according to the variations. However, it is to be noted that in other embodiments a pixel unit may be configured to comprise a plurality of pixels such as a group of adjacent pixels. That is, the processor 210 may classify all pixels into multiple groups of pixels. For each group of pixels, the processor 210 calculates variations of the brightness values (e.g. average brightness values) on the plurality of images and then to determine whether the object under test is a living body according to the variations.

Additionally, in one embodiment, before calculating the variations of brightness values, the processor 210 may employ a speckle contrast filter upon the plurality of images to preprocess the images so as to generate a plurality of processed images and then perform the above-mentioned calculation based on the plurality of processed images to calculate the variations of the brightness values of the pixel units. Each of the images is preprocessed by the speckle contrast filter to strength diversity in the image, and thus it becomes easier to calculate the variations of the brightness values of the pixel units.

The speckle contrast filter for example is arranged to perform speckle contrast computing respectively upon the plurality of images such as ten raw images, and the block/window size (e.g. M×N pixel units wherein M and N are identical/different integers) used by the speckle contrast filter can be changed under different conditions. For the ten raw images, the speckle contrast filter is arranged to enhance the contrast feature of spatial speckle contrast and/or temporal speckle contrast, to generate a plurality of processed images such as ten speckle contrast images. It is to be noted that the speckle contrast filter is optional.

In addition, the speckle contrast filter may be implemented by a speckle contrast imaging computing module/software/hardware which can be arranged to enhance the contrast of the brightness values of pixel units of the raw images to generate the processed images.

In Step 115, the processor 210 is arranged to calculate the variations based on the raw images or the processed images (i.e. speckle contrast images). For example, a variation may be calculated based on brightness values of two pixel units corresponding to the same spatial position of two adjacent raw images or two processed images; such variation may be configured as an absolute difference value of the brightness values of the two pixel units. In other words, if the processor 210 receives ten successive images, then the processor 210 can obtain nine sets of two adjacent images from the ten successive images and perform the calculations to calculate nine variations respectively based two pixel units corresponding to the same spatial position of each among the nine sets of two adjacent images.

Then, the processor 210 is arranged to accumulate all the absolute difference values, i.e. the nine variations of the same spatial position in the above example, to generate a sum for all variations among a plurality of pixel units corresponding to the same spatial position and then to generate an average value based on the sum. The average value is for all the pixel units at the same corresponding spatial position respectively within the raw/processed images. Similarly, the processor 210 can be arranged to generate multiple average values respectively corresponding to pixel units at different spatial positions respectively within the raw/processed images based on the same calculation procedure.

Further, in another embodiment, the processor 210 may employ a particular threshold to respectively compare the particular threshold with all the absolute difference values, i.e. the nine variations of the same spatial position in the above example, to decide whether an abnormal situation occurs to therefore ignore abnormal variation(s). For example, the processor 210 can detect whether the image sensor 205 is moved by comparing variation(s) with the particular threshold. If a variation is higher than the particular threshold, then the processor 210 can determine that such variation of brightness values is due to an abnormal operating situation such as the image sensor 205 is moved, and thus can ignore such variation to avoid that the average calculation is affected by the abnormal variation. The processor 210 accumulates all the variations excluding abnormal variation (s) to generate the sum of the same spatial position and then to generate an average value based on the sum.

In the embodiments, the processor 210 adopts the generated average values as variation indicators for the variations of the brightness values mentioned above. The processor 210 in Step 120 is arranged to compare the average values of multiple spatial positions with a specific average threshold to generate a statistic histogram which shows a statistic result of the number of pixel units corresponding to higher variations (compared to the specific average threshold) and a statistic result of the number of pixel units corresponding to lower variations (compared to the specific average threshold).

Then, the processor 210 is arranged to compare the number of pixel units corresponding to the higher variations with a particular number threshold. If such number of pixel units is higher than the particular number threshold, the processor 210 can determine that the object under test is a living body since images of a living body having blood flow is associated with higher image variations. Instead, if the number of pixel units is lower than the particular number threshold, the processor 210 can determine that the object under test is a non-living body since images of a non-living body not having blood flow is associated with lower image variations.

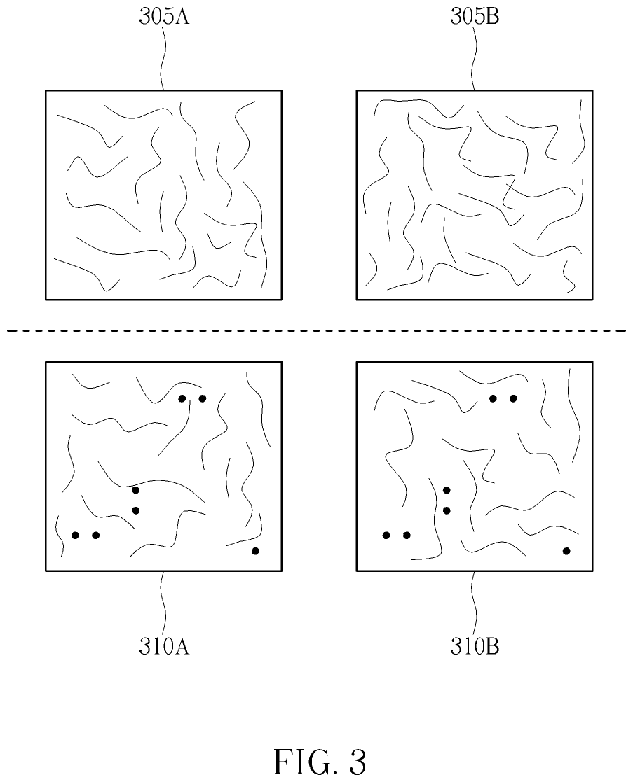

To make readers more clearly understand the concepts of the invention, FIG. 3 is an example respectively shows a difference comparison between variations of brightness values of image sensed from a living body and variations of brightness values of image sensed from a non-living body. As indicated in FIG. 3, the striations or lines of two images 305A and 305B at different timings respectively show brightness values of almost all pixels respectively in the two images are different while only few or no pixels have identical/similar brightness values, and this indicates that the images 305A and 305B are associated with a living body. If the images 305A and 305B are images of the objet under test, then the processor 210 can detect that the number of pixel units corresponding to higher variations is higher than the particular number threshold and thus can determine that the object under test is a living body.

Instead, the striations or lines of two images 310A and 310B at different timings respectively show brightness values of most pixels respectively in the two images are different while the dots show that a few pixels have identical/similar brightness values, and this indicates that the images 310A and 310B are associated with a non-living body. If the images 310A and 310B are images of the object under test, then the processor 210 can detect that the number of pixel units corresponding to higher variations is not higher than the particular number threshold and thus can determine that the object under test is a non-living body.

Further, in other embodiments, the processor 210 may compare the number of pixel units corresponding to the lower variations with another number threshold. If such number of pixel units is lower than such another number threshold, the processor 210 can determine that the object under test is a living body. Instead, if the number of pixel units is higher than such another number threshold, the processor 210 can determine that the object under test is a non-living body.

By doing so, based on the raw images such as ten raw images provided from the image sensor 205, the processor 210 is capable of quickly and accurately determining whether the object under test is a living body or a non-living body.

Additionally, in other embodiments, the processor 210 may be arranged to perform a different algorithm such as a standard deviation algorithm for the raw images or the speckle contrast images.

For example, for a particular pixel unit at a corresponding spatial position (if the particular pixel unit has only one pixel) at a first speckle contrast image or a first raw image, the processor 210 may perform the standard deviation calculation upon the pixel value of such particular pixel unit and pixel values of pixel units at the same spatial positions of other speckle contrast images (or raw images) to generate a corresponding deviation value. Similarly, the processor 210 performs the standard deviation calculation respectively upon pixel values of pixel units at different spatial positions to generate multiple deviation values for pixel units at the different spatial positions. The calculation procedure mentioned above is performed sequentially for all pixel units one by one.

The deviation values are used as the variations of the brightness values by the processor 210. For example, the processor 210 may compare the generated deviation values with a deviation threshold to generate a statistic histogram which shows a statistic result of the number of pixel units corresponding to higher deviations (compared to the deviation threshold) and a statistic result of the number of pixel units corresponding to lower deviations. For example, the processor 210 may be arranged to compare the number of pixel units corresponding to the higher deviations with a particular number threshold. If such number of pixel units is higher than the particular number threshold, the processor 210 can determine that the object under test is a living body. Instead, if the number of pixel units is lower than the particular number threshold, the processor 210 can determine that the object under test is a non-living body. In other embodiments, the processor 210 may compare the number of pixel units corresponding to the lower deviations with another number threshold. If such number of pixel units is lower than such another number threshold, the processor 210 can determine that the object under test is a living body. Instead, if the number of pixel units is higher than such another number threshold, the processor 210 can determine that the object under test is a non-living body.

It should be noted the number threshold(s) used by the calculation of the averages of variations can be identical to or different from the number threshold(s) used by the standard deviation algorithm. In addition, the deviation threshold, threshold for variations, or number threshold(s) may be predetermined or modified or can be configured by a user.

Additionally, in other embodiments, the processor 210 may be arranged to average portions or all the generated deviation values to generate a mean of deviation values. The processor 210 compares the mean with a particular mean threshold to determine whether the object under test is a living body or a non-living body. If the calculated mean is higher than the mean threshold, the processor 210 may determine that the object under test is a living body. Instead, if the calculated mean is lower than the mean threshold, the processor 210 may determine that the object under test is a non-living body.

It should be noted that the processor 210 can be arranged to calculate or estimate the variations of brightness values of the images based on other different algorithms. The calculation of the averages of variations and calculation of standard deviation are not meant to be limitations of the invention.

Further, in one embodiment, the physiological feature detection circuit 203 may be turned on/off (enabled/disabled) by the processor 210 based on the living body detection result/information of processor 210. That is, the processor 210 can stop outputting physiological features when the object under test is determined as a non-living body.

Alternatively, the physiological feature detection circuit 203 may be controlled by the other circuit such as a microcontroller based on the living body detection result/information.

Further, for power saving, the apparatus 200 can be activated or wake up periodically. Alternatively, the apparatus 200 may be always turned on as long as the electronic device 201 is enabled. Alternatively, the apparatus 200 may be turned on/off based on detection of a photo diode. For example, a photo diode may be enabled to detect environment light wherein such photo diode may be integrated within the image sensor 205 (or may be not) and can be used with the image sensor 205 to detect environment light rays. If the environment light is shut, this may indicate that an object is to be detected and thus the apparatus 200 is activated or enabled to detect whether such object is a living body. If this object is detected as a living body by the apparatus 200, the physiological feature detection circuit 203 such as a heart rate detection circuit or a blood pressure detection circuit is then activated to perform the corresponding physiological feature detection.

For detection of fake fingerprints, the conventional scheme may need to wait for at least 600 ms for the fake fingerprint detection. This, however, is not enough. Compared to the conventional scheme, for the sensing scheme/mechanism in the embodiments to successfully detect such fake fingerprint, only 100 ms is needed at most (but not limited). This significantly improves the performance of fake fingerprint detection.

Further, in other embodiments, the above-mentioned pixel unit may be configured to have a plurality of pixels. For any two adjacent images among successive images, the processor 210 may be arranged to compare pixel values of a group of pixels with pixel values of another group of pixels respectively corresponding to the same spatial position in the two adjacent images to calculate a variation of brightness values of the two groups of pixels. The variations of brightness values corresponding to a different spatial position can be calculated by comparing two groups of pixels corresponding to such spatial position respectively in any two adjacent images among successive images.

Similarly, the processor 210 may be arranged to perform the standard deviation calculation upon pixel values of groups of pixels at the same spatial area of speckle contrast images (or raw images) to generate a corresponding deviation value. These modifications also fall within the scope of the invention.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of living body detection, comprising:
capturing a plurality of images of an object under test by using an image sensor;
calculating variations of brightness values of pixel units of the plurality of images; and
determining whether the object under test is a living body according to the variations.

2. The method of claim 1, wherein the calculating step comprises:
for a particular spatial position in the plurality of images:
deriving a variation of brightness values of two pixel units corresponding to the particular spatial position respectively in any two adjacent images among the plurality of images, the deriving step being sequentially performed for pixel units corresponding to the particular spatial position respectively in each group of two adjacent images among the plurality of images to generate variations associated with the particular spatial position; and
accumulating and averaging the variations associated with the particular spatial position to calculate an average as a variation indicator for the particular spatial position.

3. The method of claim 1, wherein the calculating step comprises:
for a particular spatial position in the plurality of images:
performing standard deviation calculation upon pixel units corresponding to the particular spatial position in the plurality of images to calculate multiple deviation values for the particular spatial position; and
accumulating and averaging the multiple deviation values to calculate an average as a variation for the particular spatial position;
wherein the calculating step is sequentially performed for each spatial position one by one to calculate the variations of the brightness values of the pixel units.

4. The method of claim 1, further comprising:
employing a speckle contrast filter upon the plurality of images to generate a plurality of processed images;
wherein the calculating step is performed based on the plurality of processed images to calculate the variations of the brightness values of the pixel units.

5. The method of claim 1, wherein one of the pixel units comprises a single one pixel or a plurality of pixels.

6. The method of claim 1, wherein the determining step comprises:
comparing the variations with a specific threshold to determine whether the object under test is a living body.

7. The method of claim 6, wherein the comparing step comprises:
determining that the object under test is the living body when a number of variations corresponding to higher values is higher than the specific threshold; and
determining that the object under test is a non-living body when the number is lower than the specific threshold.

8. An apparatus having living body detection capability, comprising:
an image sensor, configured for capturing a plurality of images of an object under test; and
a processor, coupled to the image sensor, configured for calculating variations of brightness values of pixel units of the plurality of images, and for determining whether the object under test is a living body according to the variations.

9. The apparatus of claim 8, wherein the processor is configured for:
for a particular spatial position in the plurality of images:
deriving a variation of brightness values of two pixel units corresponding to the particular spatial position respectively in any two adjacent images among the plurality of images, wherein the deriving step is sequentially performed for pixel units corresponding to the particular spatial position respectively in each group of two adjacent images among the plurality of images to generate variations associated with the particular spatial position; and
accumulating and averaging the variations associated with the particular spatial position to calculate an average as a variation indicator for the particular spatial position.

10. The apparatus of claim 8, wherein the processor is configured for:
for a particular spatial position in the plurality of images:
performing standard deviation calculation upon pixel units corresponding to the particular spatial position in the plurality of images to calculate multiple deviation values for the particular spatial position; and
accumulating and averaging the multiple deviation values to calculate an average as a variation for the particular spatial position;
wherein the processor is arranged to sequentially perform the standard deviation calculation and the accumulating and averaging for each spatial position one by one to calculate the variations of the brightness values of the pixel units.

11. The apparatus of claim 8, further comprising:
a speckle contrast filter circuit, coupled to processor, configured for performing a speckle contrast calculation upon the plurality of images to generate a plurality of processed images;
wherein the processor is arranged to calculate the variations of the brightness values of the pixel units based on the plurality of processed images.

12. The apparatus of claim 8, wherein one of the pixel units comprises a single one pixel or a plurality of pixels.

13. The apparatus of claim 8, wherein the processor is arranged for comparing the variations with a specific threshold to determine whether the object under test is a living body.

14. The apparatus of claim 13, wherein the processor is arranged for:
determining that the object under test is the living body when a number of variations corresponding to higher values is higher than the specific threshold; and
determining that the object under test is a non-living body when the number is lower than the specific threshold.

15. An electronic device, comprising:
a physiological feature detection circuit;
an image sensor, configured for capturing a plurality of images of an object under test; and
a processor, coupled to the image sensor, configured for calculating variations of brightness values of pixel units of the plurality of images, and for determining whether the object under test is a living body according to the variations;
wherein the processor is arranged to disable the physiological feature detection circuit when the object under test is determined as a non-living body.

16. An electronic device, comprising:
a physiological feature detection circuit;
an image sensor, configured for capturing a plurality of images of an object under test; and
a processor, coupled to the image sensor, configured for calculating variations of brightness values of pixel units of the plurality of images, and for determining whether the object under test is a living body according to the variations;
wherein the processor is arranged to stop outputting a physiological feature when the object under test is determined as a non-living body.
